(19) United States
(12) Patent Application Publication
(10) Pub. No.: US 2020/0391203 A1
(43) Pub. Date: Dec. 17, 2020

(54) LOW-ALCOHOL JAR FOR SERIAL SALIVA AND BREATH METABOLOMICS

(71) Applicants: Roger Wallace Giese, Hanover, MA (US); Poguang Wang, Westborough, MA (US); Pushkar Kulkarni, Boston, MA (US)

(72) Inventors: Roger Wallace Giese, Hanover, MA (US); Poguang Wang, Westborough, MA (US); Pushkar Kulkarni, Boston, MA (US)

(21) Appl. No.: 16/946,278

(22) Filed: Jun. 14, 2020

Related U.S. Application Data
(60) Provisional application No. 62/860,996, filed on Jun. 13, 2019.

(51) Int. Cl. B01L 3/00; G01N 1/28; A61B 5/097; A61B 10/00; G01N 33/497; G01N 33/487; G01N 30/02; H01J 49/04; G01N 24/08

(52) U.S. Cl. CPC B01L 3/508; G01N 1/28; A61B 5/097; A61B 10/0051; G01N 33/497; G01N 33/487; A61B 2010/0009; H01J 49/0409; G01N 24/08; B01L 2300/041; B01L 2300/123; B01L 2300/06; B01L 2300/0681; G01N 30/02

(57) ABSTRACT

The present invention provides an analytical sample container including an opening of at least about 40 mm and a resealable leakproof seal. The container contains a volume of a stabilizing liquid, including an alcohol, in an amount of at least about 2 mL and less than 50% of the volume of the container. A method of biosample collection is also provided. A mixture of a biosample and a stabilizing liquid from a leakproof sealed analytical sample container is analyzed. The stabilizing liquid includes an alcohol in an amount of about 2 mL to about 50% of a volume of the container.

### CROSS-REFERENCE TO RELATED APPLICATION

This application claims the benefit of priority of U.S. provisional application No. 62/860,996, filed Jun. 13, 2019, the contents of which are herein incorporated by reference.

### BACKGROUND OF THE INVENTION

The present invention relates to containers for analytical samples and, more particularly, to containers for saliva and breath samples.

Biosamples are analyzed for a variety of reasons. Broad drug testing is needed for abuse, compliance, polypharmacy, and therapeutic drug monitoring. It is also important to conduct metabolomics since this may detect many diseases in a single test. Serial diagnostics, where a person is tested periodically, is very important, due to different individual metabolomes and a changing environment over time. Serial diagnostics may detect changes that could escape detection in a single test. Each serial pattern may be compared against earlier ones to pick up changes. Each subject thereby becomes their own control.

Furnishing blood is invasive, requires a phlebotomist, and risks soreness. Blood generally cannot be collected by a law enforcement officer in the field. Collecting the blood later means that the drug level has dropped and is no longer representative of its level in the field. Nutritional and health assessment using serial clinical monitoring of subjects is known but is done infrequently as it requires sending a phlebotomist to the subject's house to collect several tubes of blood for testing. Professional athletes do not like to furnish blood for testing of performance enhancing drugs.

Urine sample collection for performance enhancing drugs or controlled substances requires the subject be watched to guard against an adulterated or improper urine sample. Some people have great difficulty urinating when being watched.

Saliva (oral fluid) and breath (including breath aerosol) biosamples have lagged behind blood and urine for use in clinical diagnostics, including drugs, even though saliva and breath collection may be relatively noninvasive. Moreover, saliva is increasingly validated for giving the same or better information as blood or urine for many clinical diagnostic purposes. The Bridge Clinic of Substance Use Disorders at Massachusetts General Hospital routinely monitors its patients for drugs using a saliva test rather than a urine test to cut costs and time, while increasing convenience. The OraSure™ Intercept i2® cotton swab saliva collection device used has some major disadvantages that also may contribute to its failure to detect some drugs of interest.

Some police departments obtain saliva with a swab (e.g. Drager Drug Test® 5000) to help assess drug abuse. The driver or the officer rubs the swab inside the driver's mouth. While the Drager™ swab may be tested on site with a portable instrument, this takes 10 minutes; accuracy is a problem (the results do not hold up in court); the temperature needs to be above 5° C.; only a few drugs are tested; and the saliva collection device is expensive.

There is a great opportunity for serial, home-based metabolomics for clinical and drug diagnostics if saliva collection/stabilization problems are overcome. Serial home testing of saliva for clinical diagnostics has yet to be turned into practice because convenient, low cost, quick, noninvasive, reliable, comprehensive testing has not been established.

The existing techniques and devices for collecting and preserving saliva are problematic. One technique for saliva collection is to drool into a container. Another technique for collecting saliva is to adsorb it onto a cotton or synthetic polymer swab that goes into the subject's mouth. Commercial swabs are available from OraSure™ and Quantisal®. They require 3 to 15 minutes for collection of 0.5 to 1 mL of saliva. A swab may leave irritating fibers in the person's throat unless it is stabilized with a binder. Their use is irritating especially for someone with a dry mouth. One athlete in disgust bit the tip off a Quantisal® swab. Moreover, the glue necessary to keep the swab components together emits contaminants and adsorbs analytes. OraSure™ and Quantisal® oral fluid collection devices also are expensive (e.g., approximately $3.00).

The collected saliva sample is typically stabilized by freezing, making the sample difficult, if not impossible, to ship by mail. The OraSure™ and Quantisal® devices have proprietary aqueous preserving solutions. They leach chemicals into the sample and adsorb some metabolites, defeating metabolomics analysis of saliva by mass spectrometry.

Home testing devices using a sponge-swab are popular for collecting DNA from the mouth for genealogy. However, the devices defeat metabolomics because they contaminate the saliva with chemicals and with additives present to stabilize the DNA. These DNA-testing devices are far too expensive and uncomfortable for routine, serial testing for clinical diagnostics including drug testing.

As may be seen, there is a need for a simple, easy, inexpensive, and noninvasive method of collecting biosamples and preparing the biosamples for analysis, and a device therefor.

The present invention provides a wide mouth, screw cap, small, rugged, leakproof jar containing a small quantity of an alcohol. The inventive container overcomes the limitations of the prior art devices for saliva collection and analysis in the broad field of clinical diagnostics, especially metabolomics.

The invention may have big impact on health and disease, both in terms of better care and lower costs. A subject may furnish saliva in their home and then mail the inventive container at room temperature to a testing laboratory for metabolomic analysis. Home-based saliva collection may result in fewer visits to see the doctor, earlier diagnoses which may make some treatments more effective, and improved drug compliance, abuse, polypharmacy, and therapeutic drug monitoring. The inventive container may become a routine serial home-based test to monitor pregnancies for gestational diabetes and other maladies of pregnancy, such as preeclampsia.

The inventive container may be used by hospital staff as a replacement for blood draws and urine collections.

The inventive container may be used by police to test aberrant drivers for drugs of abuse. The officer may have the driver spit into the inventive container, thereby capturing the driver's drug exposure at the time of the encounter with the officer.

The inventive container may be used in any field where people impaired by drugs may injure others such as truck drivers and heavy machinery operators. Drug abuse is common among construction workers. Testing using the inventive container may become required for teachers. The inventive container may become the best way to test professional athletes for performance-enhancing drugs. The inventive container may also become the best way to monitor casino personnel for drug abuse.

### SUMMARY OF THE INVENTION

In one aspect of the present invention, an analytical sample vessel is provided comprising a container having an opening of at least about 40 mm and a resealable leakproof seal. The container contains a volume of a stabilizing liquid, comprising an alcohol, in an amount of at least about 2 mL and less than 50% of the volume of the container.

In another aspect of the present invention, a method of biosample collection is provided comprising analyzing a mixture of a biosample and a stabilizing liquid from a leakproof sealed analytical sample container. The stabilizing liquid comprises an alcohol and is present in an amount of about 2 mL to about 50% of a volume of the leakproof sealed analytical sample container.

These and other features, aspects and advantages of the present invention may become better understood with reference to the following drawings, description, and claims.

### BRIEF DESCRIPTION OF THE DRAWINGS

FIG. 1 is a cross-sectional view of a container according to an embodiment of the invention for use in sampling breath;

FIG. 2 is a cross-sectional view of the container of FIG. 1 with a leakproof storage lid sealed thereupon;

FIG. 3 is a mass spectrum of a blank (top) and a sample from Example 1 (bottom); and

FIG. 4 shows mass chromatograms (A,B,C) and a mass spectrum (D) of a sample from Example 2.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a wide mouth, screw cap, small, rugged, leakproof jar containing a small quantity of an alcohol.

The inventive container is also referred to herein as a “LOW-ALCOHOL JAR” (LAJAR). As used herein, the term jar refers to a wide-mouthed container.

The inventive container provides simple saliva collection, stabilization and sterilization in a way that is compatible with subsequent analysis even of metabolomics by mass spectrometry, as well as by other analytical techniques such as microscopy, ligand assay, ultraviolet (UV), infrared (IR), nuclear magnetic resonance (NMR), and electrophoresis. Since the LAJAR provides stabilization and sterilization of saliva at room temperature, there is no need to cool or freeze a collected saliva sample.

The wide mouth and relatively small size of the jar largely hides spitting from view since a subject partly inserts their lips into the top of the jar, enabling the subject to discreetly donate saliva. With the LAJAR, obtaining a saliva sample is no longer disgusting, irritating, slow, expensive, metabolomics-disturbing, or sloppy. There is negligible exposure of the subject to the alcohol because there is no contact of the subject's lips with the alcohol since the lips only partly enter the top of the jar; the volume of alcohol in the jar is small; and the alcohol is far removed from the lips.

The small quantity of alcohol makes it easier to mail the LAJAR, and more comfortable for a subject to collect his or her saliva in it. Because the LAJAR is leak proof for the alcohol, the LAJAR may be mailed to a subject for instant use: the subject simply unscrews the lid, spits into the jar, screws the lid back onto the LAJAR, and shakes it. The subject then may ship the LAJAR, now charged with saliva and alcohol, to a testing laboratory via regular mail or another shipping service at room temperature, since the saliva has been stabilized and sterilized at room temperature. There is no need for cooling or freezing or express mail for the saliva charged LAJAR. However, in some cases, the LAJAR may be cooled as by placement in a container of ice or dry ice. Subsequently, at the testing laboratory, the small amount of alcohol-saliva or alcohol-breath is easily subjected to analytical steps such as centrifugation, supernatant recovery, evaporation, aliquoting, filtration, electrophoresis, chromatography, and solid phase or liquid extraction before mass spectrometry analysis. The LAJAR provides a simple, universal stabilizing liquid for saliva that is compatible with metabolomics in contrast to the complex, nonuniversal, water-based solutions used to stabilize swabs to date. The LAJAR enables complete, rapid, thorough mixing of the alcohol with breath or saliva with little or no adsorption losses along with no leaching of contaminants into the sample and thereby gives a high performance for metabolomics.

The alcohol provides progressive protein precipitation for enhanced recovery of metabolites and drugs. This means that the protein precipitation is slow enough for metabolites to be extracted by the alcohol from the proteins rather than end up trapped in the protein precipitate. The alcohol instantly takes away the viscosity of the saliva to achieve complete contact of the alcohol and saliva via shaking the saliva charged LAJAR for about 5 or more times.

The LAJAR may be advantageous for collecting DNA for genetic analysis since isopropanol precipitates and stabilizes DNA. The LAJAR may be advantageous for microorganism detection. The LAJAR may also be used to receive other biosamples such as breath, breath aerosol, hair, urine, tissue, sweat, feces, tears, blood, serum, plasma, and cerebrospinal fluid.

The LAJAR may also be used to collect chemical samples in other areas of chemical analysis such as food safety, environmental monitoring/surveillance, forensics, and homeland security. In some of these cases a surface would be sampled with a swab, and the swab then would be placed into the LAJAR.

The LAJAR is suitable for use with mass tag mass spectrometry, especially utilizing mass tags possessing a quaternary amine group for increased sensitivity in case a small amount of saliva is collected from someone with a dry mouth. Of special interest are cationic xylyl (CAX) quaternary amine mass tags because of the extra boost in sensitivity they provide due to their property of anchimeric assisted neutral loss in a tandem mass spectrometer (Wang, P., Zhang, Q., Yao, Y., & Giese, R. W. (2015). Cationic Xylene Tag for Increasing Sensitivity in Mass Spectrometry. J. Am. Soc. Mass Spectrom, 26, 1713-1721, the disclosure of which is incorporated by reference).

Breath (including breath aerosol) may be collected onto a tab such as a circle of filter paper, and this tab then may be put into a LAJAR for stabilization. A tab is a good way to collect and transfer a part, or all, of many other biosamples into the LAJAR.

When saliva is collected with a swab instead of by spitting, the swab part may be cut off or broken off its stick or rod and put into the LAJAR. A nicked swab makes this convenient: just press it in the LAJAR to break off the swab. Alternatively, the subject may gargle with a solution and then dispense this solution, which then contains saliva and cells, into the LAJAR. The subject may brush their mouth with a toothbrush, sponge, or spoon to build up saliva and cells that are then dispensed into the LAJAR. The gargling solution or toothbrush or sponge may contain citric acid to help build up saliva.

The materials of manufacture are not particularly limited. Preferably the inventive container is made of plastic. Polyethylene or polypropylene are preferred since they are cheap, may be virgin, and may provide a leakproof seal between the cap and the jar with respect to the alcohol. These plastics also generally resist swelling with alcohol to avoid locking the cap on the jar when alcohol is present. Polyethylene and polypropylene are relatively nonadsorptive towards metabolites including drugs, especially the presence of alcohol. The LAJAR may comprise new, environmentally friendly plastics, which are biodegradable, if they maintain the property of preventing alcohol leakage. However, in some embodiments, the composition of the container may not be plastic due to incompatibility with the solvent. In these cases, a glass jar, for example, may be preferred, although glass is heavier and risks breakage.

The inventive container may have a capacity of about 1 to greater than about 4 ounces, with about a 40-60 mm opening, a wide base with a diameter at least as wide as the opening, and a height of about 40 to 70 mm. The LAJAR preferably is about 2-3 ounces in capacity. Dimensions within this range permit the subject to insert their lips into the device and gently and discreetly spit saliva into it without their lips contacting the alcohol. The wide width of the LAJAR, usually having a flat base, makes it easy to stand up and resist falling over. The LAJAR may be cylindrical and may have a conical shape in some cases. A larger LAJAR may be employed as needed (e.g. 4 oz or larger). For a child, a smaller LAJAR may be used, or a usual LAJAR but with a smaller opening. Even a one oz. jar may be used to practice this invention if it does not leak isopropanol, although a smaller jar may be more awkward to use. For example, 43- and 53-mm openings are available in a Bio-Tite® jar. A Samco™ Bio-Tite® specimen jar with a capacity of 2-3 oz. may be suitable for the invention. A similar Bio-Tite® jar from Thermo™, #02 1038, also may be used as a LAJAR. This jar has a 3 oz capacity, an opening of 53 mm, and comprises virgin polypropylene. Other suitable containers may include the Bio-Tite® 60 mL jar from Thermo Scientific®, model 13-711-64, which has a 48 mm opening and comprises virgin polypropylene. Other Bio-Tite® jars also may be used as a LAJAR. One suitable container is the Bio-Tite® #02 0039 jar from Fisher Scientific®.

Preferably, the inventive container has the following features: virgin polypropylene, wide mouth (about 43 mm opening), about 3 oz. capacity, does not leak isopropanol, is relatively small, and does not swell shut when it contains isopropanol. These features provide a sample container with a long shelf life, probably many years.

The stabilizing liquid of the present invention comprises at least an alcohol and may be present in an amount of at least about 2 mL and up to about 50% of the container volume. Preferably, the stabilizing liquid in the container is about 2 mL.

The invention may contain an amount of alcohol up to about 50% of its volume for some applications. Preferably the quantity of alcohol is in the range of about 2-3 mL. In some aspects, the volume of alcohol is less than 10% of the volume of the jar. A larger LAJAR may contain a larger volume of alcohol, such as 3-5 mL. The small volume of alcohol is suitable for a single spit sample, giving a percent of alcohol in the about 50 to about 90% range that is optimal for sterilization, and gives instant stabilization of the saliva at room temperature. The small volume enables convenient use by the subject, facilitates mailing, lowers cost, and provides a low weight for the LAJAR.

The alcohol in the LAJAR may be selected from the group consisting of ethanol, methanol, propanol, isopropyl alcohol, n-butanol, sec-butanol, t-butyl alcohol, propylene glycol, benzyl alcohol, and combinations thereof. Preferably the alcohol comprises isopropyl alcohol, but other alcohols from the preceding group may be present. Isopropanol is the preferred solvent for several overlapping reasons: (1) it denatures and penetrates proteins progressively to fully or highly extract protein-complexed small molecules; (2) it is miscible with water; (3) is considered safe (it is the major component of rubbing alcohol); (4) it is inexpensive; (5) it has a recognizable, friendly odor which makes it consumer-acceptable while also keeping the consumer from drinking it; (4) it does not leak, either as liquid or vapor, from sealed polypropylene and/or polyethylene containers; (5) it is sufficiently volatile for easy evaporation later at the laboratory conducting mass spectrometry analysis; (6) a low volume of isopropanol stabilizes and sterilizes saliva and also makes subsequent metabolomics easier; (7) unlike primary alcohols, is not susceptible to oxidation to form reactive aldehydes; (8) it is compatible with mass tag derivatization for enhanced analysis of saliva by mass spectrometry; and (9) it lowers the viscosity of saliva and breath aerosol, giving a saliva-alcohol or breath-alcohol mixture that is nonviscous and therefore easy to handle, providing complete mixing with a few shakes. The alcohol may be partly aqueous. In some embodiments the alcohol is present in the inventive container before saliva or breath is added. In other embodiments, saliva or breath may be collected first, and alcohol may be added immediately thereafter.

Other organic solvents may be used, alone or in combination with an alcohol, water, or alcohol/water combination. These solvents may be selected from the group consisting of: ethers, nitriles, esters, carboxylic acids, ketones, alkanes, alkenes, aromatics, amides, phenols, haloalkanes, and haloaromatics. For example, one or more solvents may be selected from the group consisting of: t-butylmethylether, acetonitrile, ethyl acetate, acetic acid, acetone, cyclohexane, heptane, toluene, dimethyformamide, phenol, dichloromethane, and chlorobenzene.

Acids may be present such as hydrochloric acid or phosphoric acid. Bases may be present such as ammonium hydroxide or sodium hydroxide. Buffers may be present such as borate, carbonate, acetate, phosphate, or TRIS. Enzymes may be present such as trypsin or a nuclease.

Additives may be present such as a chelating agent, surfactant, isotopic internal standard, chromatographic particles, acid, salt, or a dye. One purpose of a salt when present is to absorb the water of the saliva.

In an embodiment, a plastic bag containing alcohol is provided. In this embodiment, the bag may be conveniently sealed and unsealed and, when sealed, the bag does not leak alcohol. For example, a biohazard or other zip-lock bag may be used. This embodiment may be referred to herein as a LAJAR-BAG.

In an embodiment, the cap of the LAJAR may be lined with a metal foil.

In an embodiment, a pad of porous material may be attached to the inside lid of the LAJAR for breath collection. Alternatively, a parallel, spaced stack of membranes or filter papers may be attached perpendicularly or parallel to the lid or bristles may be attached to the lid. The pad may be manufactured from a material selected from the group consisting of cotton, polypropylene, stainless steel, Teflon®, glass wool, nylon, rayon, copper, aluminum, teabag, and a porous particulate material such as silica or bonded silica, and a mesh containing one or more of the foregoing materials. The pad may be attached to the inside of the lid by any suitable method, such as stapling, clipping, embedding, sewing, hooking, screwing, grabbing, tying, or gluing. The pad may be wetted by alcohol in the jar by shaking the jar or by being in contact with fumes from alcohol contained within the jar. An aqueous alcohol solution is preferred to minimize toxicity. To use, the user may simply unscrew the lid, breathe on the pad, and screw the lid back onto the jar. The lid-attached pad on a LAJAR also may be used to extract the precipitated macromolecules when saliva contacts the alcohol, so that shaking such a jar provides sample cleanup, separating small molecules from large molecules. The pad also may have adsorption properties such as ion exchange. Adding extra water or some solvent to the saliva-containing jar may also enhance this process, for example by increasing the opportunity for nonpolar extraction of saliva chemicals onto the pad. Using a small pad may focus such extracted chemicals.

In an embodiment, a collection element is contained in a LAJAR and may be removed from the LAJAR with a forceps or tongs or pliers for exposure to breath, then returned to the jar. The collection element either may be allowed to partly dry, as by patting with a tissue, before exposure to breath, or may be exposed to breath while still wet. The collection element may be a filter paper, or other tab, or mesh and may be reinforced with a mesh or screen, such as one made of metal or a paper clip, for easier handling. The collection element may be stapled to the mesh or screen.

In an embodiment, two jars may be used for breath collection, wherein only the second jar is a LAJAR. The first jar may contain a trapping material for breath such as a tab or mesh to avoid contamination of the material by the environment. To collect breath, the user may remove the material from the first jar, breathe on it, and then put it into the second jar.

In an embodiment, the inventive jar further includes a straw. A Straw-Entrained or -Attached Plug (SEAP) of fabric, frit, fiber, particles, electret, or other porous material may be present at, in, or up the jar end of the straw, and perhaps touched to the liquid periodically, to enhance breath trapping. The user may suck up some liquid into the SEAP straw in-between breaths. Useful as well is a plugged straw at the jar end, where the wall of the straw is porous. If there were a SEAP or porous wall at the jar-end of the straw, between breaths the liquid would spontaneously re-wet this part, to enhance trapping of breath chemicals.

In an embodiment, the container may comprise an exit tube which may contain charcoal or some other adsorbent to trap the exit vapor.

In an embodiment, fabric such as cotton or glass wool may be present in the LAJAR, especially when it is wetted by the trapping liquid, to enhance breath trapping.

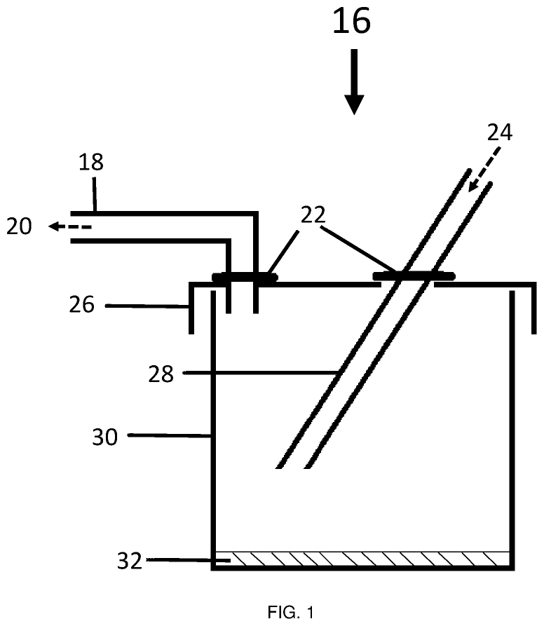

Referring to FIGS. 1-4, FIG. 1 illustrates a method of collecting breath for metabolomics including broad detection of drugs, using an embodiment of the inventive container, where the breath chemicals are trapped in a liquid contained therein. Preferably the liquid is isopropanol. As seen, breath 24 is delivered by a person through a straw 28 that is mounted through a sample lid 26 on a LAJAR 16 so that the breath flows over the surface of the liquid 32 prior to exiting out through an exit tube 18. Both the straw 28 and the exit tube 18 may be secured within the sample lid 26 by retainers 22. The exit tube 18 may direct the exit vapor 20 from the trapping liquid 32 away from a person's mouth. Alternatively, the subject may exhale into a tube (not shown) that connects to the straw 28 and thereby be more remote from the LAJAR. Although the straw 28 is shown in FIG. 1 elevated above the liquid, the straw 28 may be lowered into the liquid so that breathing (i.e., blowing through the straw 28) bubbles breath through the liquid.

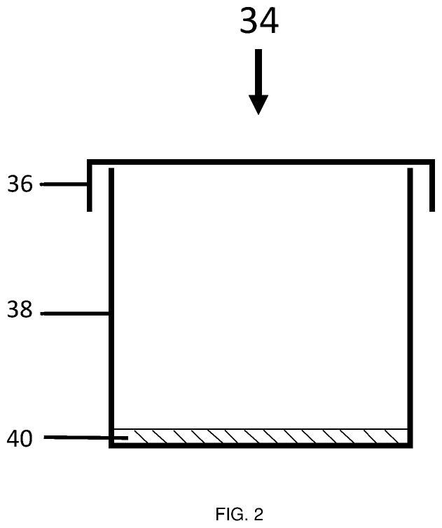

The sample lid may be removed from the jar 38 and replaced with a nonleaking shipping and/or storage lid 36 as illustrated in FIG. 2, so the sealed LAJAR 34 may be shaken, mixing the liquid and breath 40, and subsequently mailed to an analytical laboratory.

### Example 1

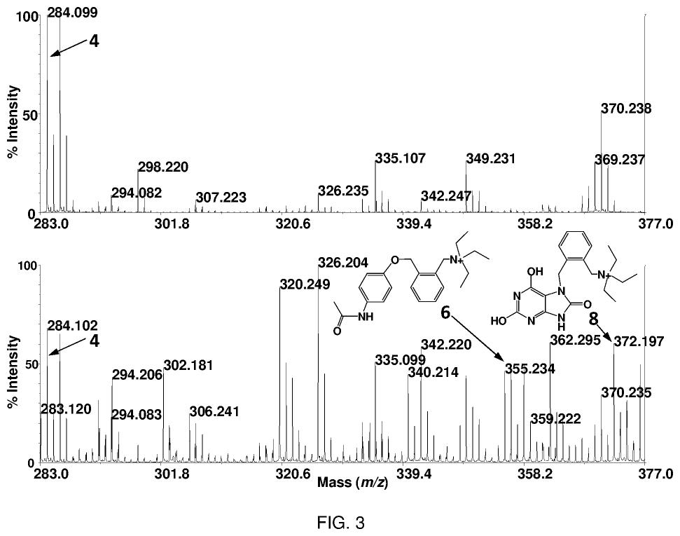

A #02 0039 plastic jar from Fisher Scientific® was charged with 2 mL of high-performance liquid chromatography (HPLC)-grade isopropanol from the same company. A male subject had taken an acetaminophen (i.e., Tylenol®) tablet two hours earlier. After sucking for 20 seconds with his mouth closed to enhance and bring his saliva to the front of his mouth, the subject unscrewed the lid from the LAJAR, put his lips partly into it, gently spit into it, and screwed the lid back on. The LAJAR was shaken for a few seconds and wrapped in aluminum foil to keep it dark. Later, 1 mL of this sample was centrifuged at 13,000 rpm in Fisher Scientific® Accuspin® Micro 17 for 3 min. Ten μL of supernatant was dried in SpeedVac®, and mixed with 40 μL of cationic xylyl-bromide (CAX-B), 1 mg/mL, with triethylamine, 1 μL/mL, in acetonitrile (ACN):water 1:1. The reaction mixture was kept at 37° C. for 14 hours, evaporated in SpeedVac®, and redissolved in 40 μL of 10% ACN in water, and 1 μL was analyzed by a matrix-assisted laser desorption/ionization (MALDI)-time-of-flight (TOF)-mass spectrometer (MS), giving the data shown in FIG. 3, including a peak for Tylenol® 6. Many peaks for metabolites are also observed, including a peak for the metabolite uric acid 8. A peak for the CAX-B mass tag 4 at about 284 mass units is present in both the blank (top) and the saliva sample results (bottom).

### Example 2

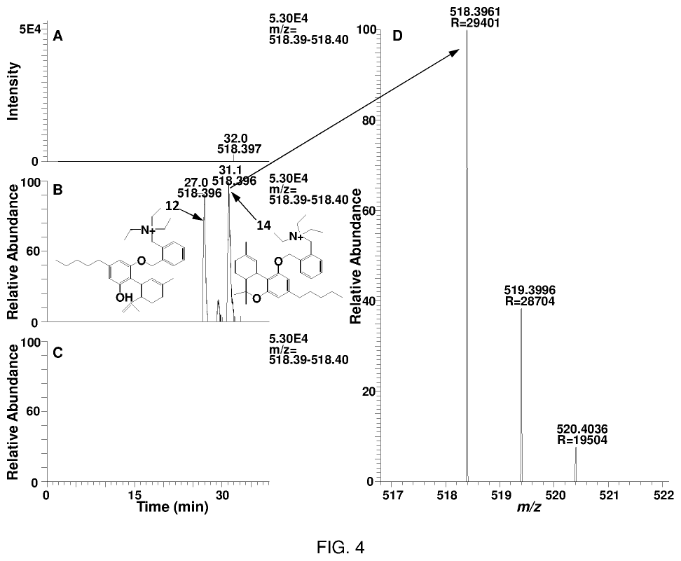

A subject spit into a LAJAR containing 2 mL of isopropanol, giving a blank sample at time zero. The subject then took 8 puffs of a marijuana cigarette and furnished further single spit deposits into a series of LAJARs at other time points, one spit per LAJAR, including spits at 1 hour and 16 hours. These samples were stored in the dark at room temperature for 6 days. From the blank, 1-hour, and 16-hour samples was taken 50 μL of the saliva and isopropanol mixture. These samples were centrifuged at 13,000 rpm for 3 min; 10 μL of supernatant was dried in a SpeedVac®; 20 μL of CAX-B at 1 mg/mL with triethylamine at 1 μL/mL in 50% ACN was added to the dried sample; the mixture was kept at 37° C. for 10 h. After evaporation in a SpeedVac®, each sample was dissolved in 25 μL of 10% ACN and 0.1% formic acid and centrifuged at 13,000 rpm for 3 min, 10 μL of supernatant was injected into a liquid chromatography (LC)/Orbitrap® MS (1×250 C18, 40 μL/min from 6 to 95% ACN in 20 min). Results are shown in the selected ion monitoring (SIM) mass chromatograms of FIG. 4. As seen, no cannabinoids were detected in the blank A. However, six cannabinoids were detected in the one-hour sample cannabidiol (CBD), delta-9-tetrahydrocannabinol (Δ⁹-THC), cannabinol (CBN), delta-9-tetrahydrocannabinolic acid (Δ⁹-THCA), THCtriol, and an unknown cannabinoid; the detection of two of these, CBD 12 and THC 14, is shown in inset B of this figure. These two cannabinoids were no longer detected in the 16-hour sample, as seen in inset C. A small amount of just one cannabinoid, CBD, was detected in the 16-hour sample (data not shown). Inset D shows the molecular ions for the peak at 31.1 minutes as indicated in the mass spectrum of the one-hour sample of inset B.

### Example 3

Eleven pregnant women obtaining OB-GYN care at Beth Israel Deaconess Medical Center, Harvard Medical School, each furnished one spit of saliva into a separate LAJAR. The eleven LAJARS were immediately shaken to sterilize and stabilize the saliva metabolites. Five of the women had gestational diabetes and six exhibited blood glucose levels in the normal range. One of the samples (from a “normal” patient) was lost. The samples were tested in a blinded way for metabolomics by CAX mass tag mass spectrometry as above in example 2. Principle component analysis (PCA) of the metabolomic data divided the samples into two groups: those with closely clustered PCA values (6 samples) and those with remote PCA values relative to the former (4 samples). Of the 5 subjects with gestational diabetes, 4 were in the latter group. This was an exciting result since gestational diabetes is a major disease and early diagnosis may enable treatment that reduces morbidity and mortality.

It should be understood, of course, that the foregoing relates to exemplary embodiments of the invention and that modifications may be made without departing from the spirit and scope of the invention as set forth in the following claims.

What is claimed is:

1. An analytical sample vessel comprising:
a. a container having an opening of at least about 40 mm; and
b. a resealable leakproof seal;
wherein said container contains a volume of a stabilizing liquid, comprising an alcohol, in an amount between at least about 2 mL and less than about 50% of the volume of the container.

2. The analytical sample vessel of claim 1, wherein the alcohol is selected from the group consisting of: ethanol, methanol, propanol, isopropyl alcohol, and combinations thereof.

3. The analytical sample vessel of claim 1, wherein the vessel comprises a jar and a lid, at least one of which comprises a material selected from the group consisting of: polyethylene; polypropylene; glass; and a biodegradable plastic resistant to alcohol leakage.

4. The analytical sample vessel of claim 3, wherein the lid is effective to provide the leakproof seal when attached to the jar.

5. The analytical sample vessel of claim 1, further comprising a sample lid having a straw and an exit tube.

6. The analytical sample vessel of claim 1, further comprising a sample collection material selected from the group consisting of: filter paper, a porous pad; a stack of membranes; bristles; mesh; a collection tab; and adsorbent.

7. The analytical sample vessel of claim 1, wherein the container is a resealable plastic bag.

8. A method of biosample collection comprising:
analyzing a mixture of a biosample and a stabilizing liquid from a leakproof sealed analytical sample container, wherein the stabilizing liquid comprises an alcohol in an amount of about 2 mL to about 50% of the volume of the leakproof sealed analytical sample container.

9. The method of claim 8, wherein the biosample is selected from the group consisting of: saliva, breath, breath aerosol, hair, urine, tissue, sweat, feces, tears, blood, serum, plasma, and cerebral spinal fluid.

10. The method of claim 8, further comprising a step of collecting the biosample selected from the group consisting of: swabbing a body part of a subject; collecting breath on a sample collection element; gargling and spitting; brushing a subject's oral cavity and spitting; and scraping the subject's oral cavity and spitting.

11. The method of claim 8, wherein the step of analyzing comprises at least one analysis selected from the group consisting of: mass spectrometry, microscopy, ligand assay, ultraviolet, infrared, nuclear magnetic resonance, electrophoresis and chromatography.

12. The method of claim 8, wherein prior to the step of analyzing, the mixture of the biosample and the stabilizing liquid are subjected to preparation selected from the group of: reaction with a mass tag; supernatant recovery; aliquoting; evaporation; filtration; centrifugation; extraction; electrophoresis; chromatography; and a combination thereof.

13. The method of claim 8, wherein prior to the step of analyzing, the leakproof sealed analytical sample container is stored and/or transported at room temperature.
